(19) United States
(12) Patent Application Publication
(10) Pub. No.: US 2016/0038161 A1
(43) Pub. Date: Feb. 11, 2016

(54) PATIENT SPECIFIC INSTRUMENTS

(71) Applicant: Luke Andrew Gibson, Southaven, MS (US)

(72) Inventor: Luke Andrew Gibson, Southaven, MS (US)

(73) Assignee: SMITH & NEPHEW, INC., Memphis, TN (US)

(21) Appl. No.: 14/116,631

(22) PCT Filed: Apr. 25, 2012
(86) PCT No.: PCT/US2012/034957

§ 371 Date: Jul. 14, 2014

Related U.S. Application Data
(60) Provisional application No. 61/484,066, filed on May 9, 2011.

(51) Int. Cl. A61B 17/17; A61B 17/16; A61F 2/30; A61F 2/46

(52) U.S. Cl. CPC A61B 17/1746; A61F 2/4609; A61B 17/1666; A61F 2/30942; A61F 2002/30952; A61F 2002/30948; A61B 19/50

(57) ABSTRACT

Disclosed herein are systems, devices, and methods for orthopedic surgical guides adapted to the anatomy of a patient and configured to align a bone shaping device or acetabular implant in a predetermined orientation. Certain embodiments constitute alignment guides for conducting hip surgery on a specific patient. Such guides include a first or inner surface structured to fit in a predetermined configuration relative to a portion of a patient's anatomy based on topography data of the patient's anatomy, a receiving surface configured to receive or constrain a bone shaping device or acetabular implant, and a reference surface with an alignment site that directs placement of the orthopedic device in a predetermined orientation relative to the patient's anatomy when the first surface is in the predetermined configuration.

### CROSS-REFERENCE TO RELATED APPLICATIONS

This application claims the benefit of U.S. Provisional Application No. 61/484,066, filed May 9, 2011, which is hereby incorporated by reference herein in its entirety.

### RELATED FIELDS

The disclosure relates generally to instruments for conducting hip and other surgeries and, more particularly, instruments designed for a patient's specific anatomy.

### BACKGROUND

Joints undergo degenerative changes due to a variety of reasons. When joint degeneration becomes advanced or irreversible, it may become necessary to replace the natural joint with a prosthetic joint. Artificial implants, including hip joints, shoulder joints, and knee joints are widely used in orthopedic surgery. Specifically, hip joint prostheses are common. The human hip joint acts mechanically as a ball and socket joint, wherein the ball-shaped head of the femur is positioned within the socket-shaped acetabulum of the pelvis. Various degenerative diseases and injuries may require replacement of all or a portion of a hip or other joints using prosthetic implants constructed of metals, ceramics, plastics, or other synthetic materials.

Joint preparation for reconstruction and placement of a prosthetic implant typically requires re-shaping the joint by cutting, reaming, drilling, or other surgical procedures. Proper shaping is critical for successful joint reconstruction and fixation of prosthetic implants. However, since each patient has a unique anatomy, the shaping process poses a significant challenge for surgeons, particularly in cases of substantial bone degeneration and defects. Surgical guides for the cutting, reaming, and drilling steps can help surgeons properly and accurately prepare the joint. Surgical guides that are adapted to individual patient anatomy can be particularly valuable for effective joint reconstruction.

### SUMMARY

Disclosed herein are systems, devices, and methods for orthopedic surgical guides adapted to the anatomy of a patient and configured to align a bone shaping device or acetabular implant in a predetermined orientation. Certain embodiments constitute alignment guides for conducting hip surgery on a specific patient. Such guides include a first or inner surface structured to fit in a predetermined configuration relative to a portion of a patient's anatomy based on topography data of the patient's anatomy, a receiving surface configured to receive or constrain a bone shaping device or acetabular implant, and a reference surface with an alignment site that directs placement of the orthopedic device in a predetermined orientation relative to the patient's anatomy when the first surface is in the predetermined configuration.

In certain embodiments, the inner surface has a contour that conforms to at least a portion of a specific patient's anatomy. This portion of the guide is generally a negative or complement of the topography of portions of the pelvic bone, cartilage, or other anatomy of a specific patient in which the guide fits. In some embodiments, such patient-matched surfaces may be formed using topography data obtained from the specific patient by imaging devices and using computer aided design and manufacturing techniques, although, in other embodiments, other technologies may be used to define and form the patient-matched surfaces.

The reference surface has an alignment site for directing placement of the orthopedic device in a predetermined orientation relative to the patient's anatomy. In certain embodiments the position of the alignment site is based on the topography data of the patient. The placement of the alignment site in a predetermined position aligns the guide in a predetermined configuration to guide an orthopedic device, such as a bone reamer, impactor, or implant. The alignment site may include indicia or an indicator that is scored, marked, or integrally formed with respect to the reference surface. This indicator may uniquely identify a patient. In certain embodiments, the indicator identifies a predetermined anatomical location for placement of the surgical guide.

The guide includes a reference surface that, in certain embodiments, lies in a plane parallel to a surface of the orthopedic device when the orthopedic device is placed in the predetermined orientation. The reference surface may include one or more steps, where each step corresponds to a different predetermined depth of the orthopedic device. For example, a first step may correspond to a predetermined depth for positioning a reamer and a second step may correspond to a predetermined depth for positioning an implant.

The surgical guide is configured for placement in a predetermined configuration relative to the anatomy of a specific patient. The predetermined configuration may include a predetermined angle of the orthopedic device relative to the patient's anatomy, for example varus or valgus angles. The alignment site may correspond to predetermined angles for guiding the orthopedic device, such as a reamer, impactor, or implant. When the surgical guide is properly placed, the guide allows a user, such as surgeon, to accurately place an orthopedic device in a predetermined orientation relative to the anatomy of a specific patient.

The receiving portion may be configured to physically and/or visually guide a bone shaping device or acetabular implant into the pelvis in a specified spatial location and orientation along a given axis relative to the pelvis. The receiving surface may correspond to predetermined angles for guiding the orthopedic device, such as a reamer, impactor, or implant. In certain embodiments, the receiving surface is concave.

In some embodiments, the alignment guide is configured to receive reference pins or other components to secure the alignment guide to the patient's anatomy. In at least some embodiments, the alignment guide provides retractor functionality to maintain the incision site open.

The orthopedic surgical guide can be structured in size and shape suitable for minimally invasive surgeries for anterior or other approach hip surgery. The size, shape and other aspects also allow the guide to be easily introduced to and positioned on the patient's pelvis. Other embodiments include methods of making and methods of installing such a guide.

An alignment guide is provided herein for conducting orthopedic surgery, such as hip surgery, on a specific patient. In certain implementations, the guide includes: an inner surface configured in shape to conform or be complementary to at least a portion of the specific patient's anatomy, the inner surface formed at least in part according to topography data of portions of the specific patient's anatomy; a receiving portion configured to receive or constrain a bone shaping device or an acetabular implant; a plurality of openings, each openings configured to receive a reference pin; and a reference surface that is generally planar in shape, the reference surface having an alignment site or indicator configured to serve as a visual and/or physical reference for guiding the bone shaping device or reamer.

An alignment guide is provided herein for conducting orthopedic surgery, such as hip surgery, on a specific patient. In certain implementations, the guide includes: an inner surface configured in shape to conform to at least a portion of the specific patient's anatomy, the inner surface formed at least in part according to data defining topography of portions of the specific patient's anatomy; a receiving portion forming a substantially concave surface, the receiving portion configured to receive a bone shaping device or an acetabular implant; a generally planar reference surface abutting at least a portion of the receiving portion to form a rim that extends at least partially around the receiving portion, the reference surface having an alignment site or indicator configured to serve as a visual and/or physical reference for guiding the bone shaping device or reamer; and a plurality of openings formed in the guide and oriented substantially parallel to the plane of the reference surface, each opening configured to receive a reference pin.

A method of installing an alignment guide is provided herein for conducting orthopedic surgery, such as hip surgery, on a specific patient. In certain implementations, the method includes: providing a surgical guide having a first surface, receiving surface, and reference surface with an alignment site that directs placement of the orthopedic device in a predetermined orientation relative to the patient's anatomy, wherein the first surface is structured to fit in a predetermined configuration relative to a portion of a patient's anatomy based on topography data of the patient's anatomy; placing the first surface on the patient anatomy in the predetermined configuration; mating the orthopedic device with the receiving surface; and aligning a portion of the orthopedic device with the reference surface. The method may also include creating an incision in the patient to access the patient's hip. In certain implementations, the guide has at least one opening and the method includes inserting a pin into the opening to secure the guide to bone or tissue. In some embodiments, the bone shaping device is guided such that a reference surface on the bone shaping device is substantially co-planar or substantially parallel to the reference surface of the alignment guide.

Methods are also provided for making an alignment guide for conducting orthopedic surgery, such as hip surgery, on a specific patient, which may include: obtaining data indicative of a patient's anatomy; creating a model of the patient's anatomy from the data; determining a suitable position for an orthopedic device relative to the model; forming a first surface structured fit in a predetermined configuration relative to the patient's anatomy based on the model; forming a receiving surface structured to contact a portion of an orthopedic device; forming a reference surface with an alignment site that directs placement of the orthopedic device in a predetermined orientation relative to the patient's anatomy. In certain implementations, the method includes forming at least one opening, based on the model, that is structured to receive a fastener, such as a pin. In certain implementations, the method includes forming a contour on the first surface that is complementary to a portion of the patient's anatomy. In some embodiments, a plane defining the position and/or orientation of the reference surface will be substantially perpendicular to an axis defining desired inclination and/or anteversion angles of a hip implant. In some embodiments, the plane defining the reference surface will relate to a desired insertion depth of the hip implant. In some embodiments, the plane defining the reference surface will be positioned and oriented to reflect a desired position and/or orientation of a rim or other reference surface on the bone shaping device and/or implant. In some embodiments, the axis and/or plane will be defined using the data relevant to the specific patient's pelvis.

An alignment guide is provided herein for conducting orthopedic surgery, such as hip surgery, on a specific patient. In certain implementations, the guide includes an inner surface means configured in shape to conform to at least a portion of the specific patient's pelvic anatomy, the inner surface means formed at least in part according to computer-obtained data mapping at least portions of the specific patient's pelvic anatomy; a receiving means configured to receive a bone shaping device or an acetabular implant; an outer surface means including a plurality of openings, each opening configured to receive a reference pin; and a reference means that is generally planar in shape, the reference means configured to serve as a visual and/or physical reference for guiding the bone shaping device or reamer.

According to some embodiments the alignment guide includes a guide wherein the computer obtained data defining topography of portions of the specific patient's pelvic anatomy includes data defining topography of at least portions of cartilage. According to some embodiments, the alignment guide includes a guide wherein the computer obtained data defining topography of portions of the specific patient's pelvic anatomy includes data defining topography of at least portions of bone.

According to some embodiments the alignment guide includes a guide wherein the reference surface forms a stepped surface. According to some embodiments the alignment guide includes a guide wherein the reference surface is arcuate and surrounds at least a portion of the receiving portion. According to some embodiments the alignment guide includes a guide wherein the receiving portion is generally concave. According to some embodiments the alignment guide includes at least one reference pin oriented substantially parallel to the plane of the reference surface. According to some embodiments the alignment guide includes a guide wherein the reference surface abuts a portion of the receiving portion to form a rim that extends at least partially around the receiving portion.

According to some embodiments the alignment guide includes a guide wherein at least a portion of the inner surface is formed using computer aided machining, controlled at least in part by the data defining topography of portions of the specific patient's pelvic anatomy. According to some embodiments the alignment guide includes a guide wherein at least a portion of the inner surface is formed using three dimensional printing controlled at least in part by the data defining topography of portions of the specific patient's pelvic anatomy.

Advantages of certain, non-limiting embodiments include alignment guides (and systems and/or methods for making and using them) for the pelvis that allow accurate positioning of a bone shaping device in the pelvis, in either or both location and angular orientation, for hip reaming or other hip surgery; allow accurate positioning of an acetabular implant in the pelvis, in either or both location and angular orientation, for hip replacement or other hip surgery; can feature inner surfaces that are prepared in a patient specific manner, according to data obtained from a specific patient, for positioning and orienting a bone shaping device or acetabular implant for hip replacement, hip reaming, or other hip surgery; can be prepared using computer aided design and/or computer aided manufacturing techniques and patient specific data, in addition to, if desired, input or data from the surgeon; can be made sufficiently small and unobtrusive to allow for use in minimally invasive surgery, including anterior and other approach hip surgery, but yet can retain their position in the pelvis once placed in position for accurate location, orientation, and angle of a bone shaping device or acetabular implant.

Further features and advantages of at least some of the embodiments, as well as the structure and operation of various embodiments, are described in detail below with reference to the accompanying drawings.

### BRIEF DESCRIPTION OF THE DRAWINGS

The accompanying drawings, which are incorporated in and form a part of the specification, illustrate different embodiments and together with the description, serve to explain the principles of such embodiments. In the drawings:

FIG. 1 illustrates an embodiment of a patient specific alignment guide positioned on a pelvis.

FIGS. 2A-2D illustrate an embodiment of a patient specific alignment guide.

FIG. 3 illustrates an embodiment of a patient specific alignment guide with angle indicators.

FIGS. 4A-4B illustrate an embodiment of a patient specific alignment guide with a reference surface having a plurality of raised surfaces.

FIGS. 5A-5C illustrate an embodiment of a patient specific alignment guide with an acetabular implant.

FIGS. 6A-6B illustrate an embodiment of a patient specific alignment guide positioned on a pelvis.

FIG. 7 illustrates an embodiment of a patient specific alignment guide positioned on a pelvis through an anterior-approach incision.

FIGS. 8A-8B illustrate a bone shaping device used during surgery.

FIG. 9 is a flow diagram depicting a process for making and using a patient specific alignment guide to perform surgery.

### DETAILED DESCRIPTION

To provide an overall understanding of the systems, devices, and methods described herein, certain illustrative embodiments will be described. Although the embodiments and features described herein are specifically described for use in connection with orthopedic hip procedures, it will be understood that all the components, connection mechanisms, adjustable systems, manufacturing methods, and other features outlined below may be combined with one another in any suitable manner and may be adapted and applied to devices and implants to be used in other surgical procedures, including, but not limited to hip arthroplasty, knee arthroplasty, spine arthroplasty, cranio-maxillofacial surgical procedures, shoulder arthroplasty, as well as foot, ankle, hand, and other extremity procedures.

Disclosed herein are systems, devices, and methods for orthopedic surgical guides adapted to the anatomy of a patient and configured to align a bone shaping device or acetabular implant in a predetermined orientation. Certain embodiments constitute alignment guides for conducting hip surgery on a specific patient. Such guides include a first or inner surface structured to fit in a predetermined configuration relative to a portion of a patient's anatomy based on topography data of the patient's anatomy, a receiving surface configured to receive or constrain a bone shaping device or acetabular implant, and a reference surface with an alignment site that directs placement of the orthopedic device in a predetermined orientation relative to the patient's anatomy when the first surface is in the predetermined configuration.

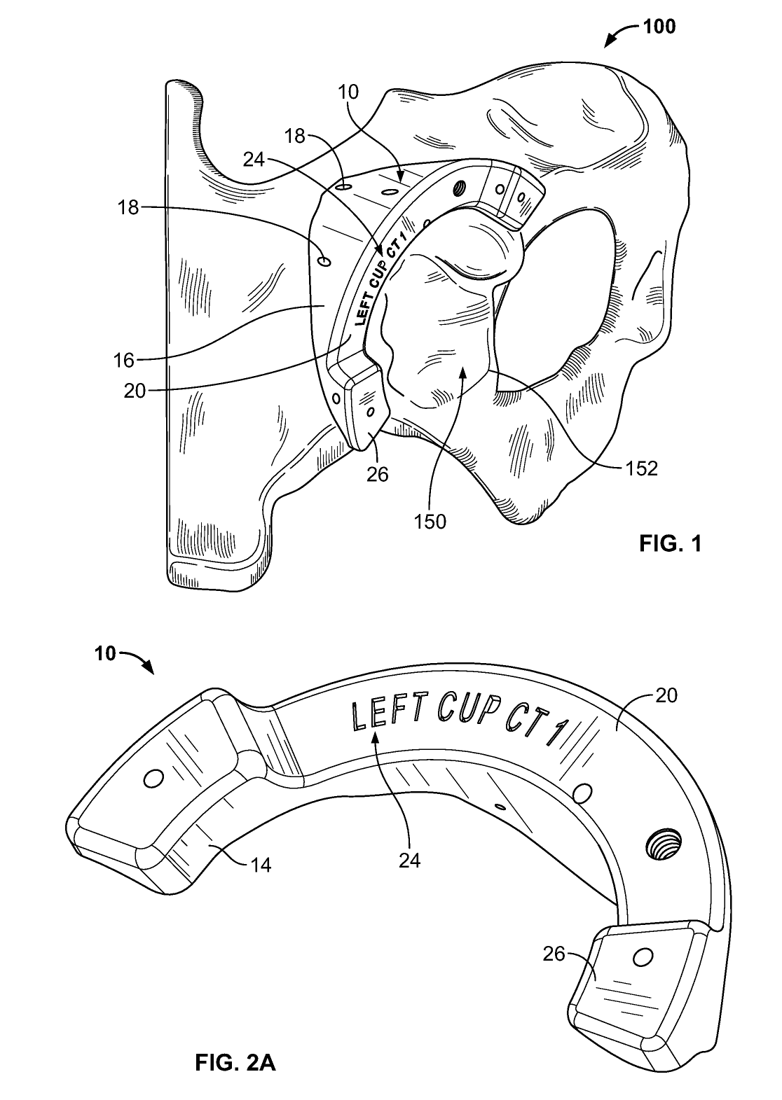

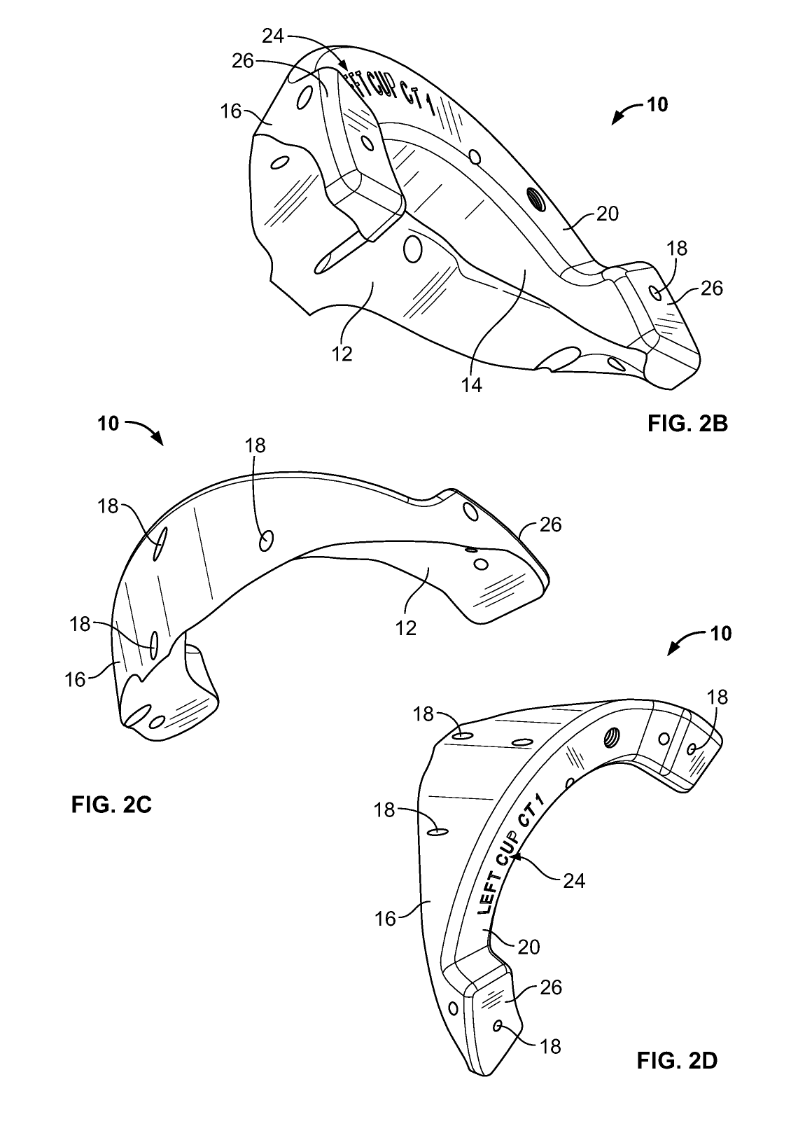

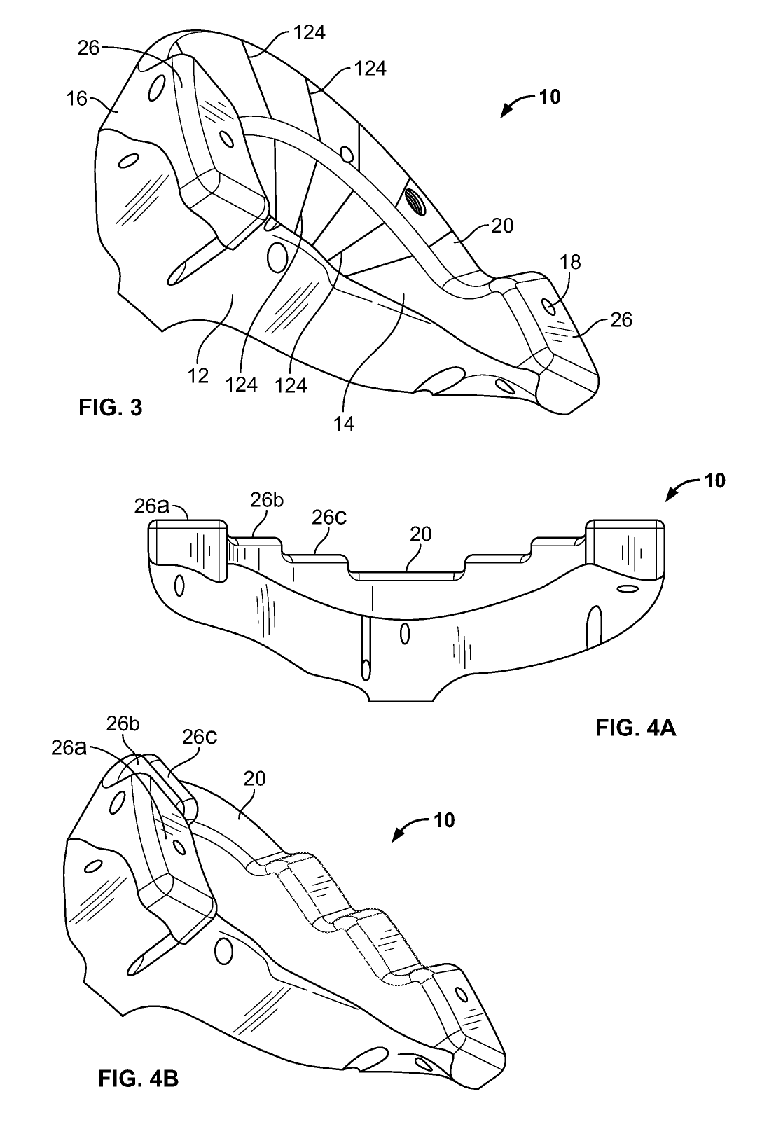

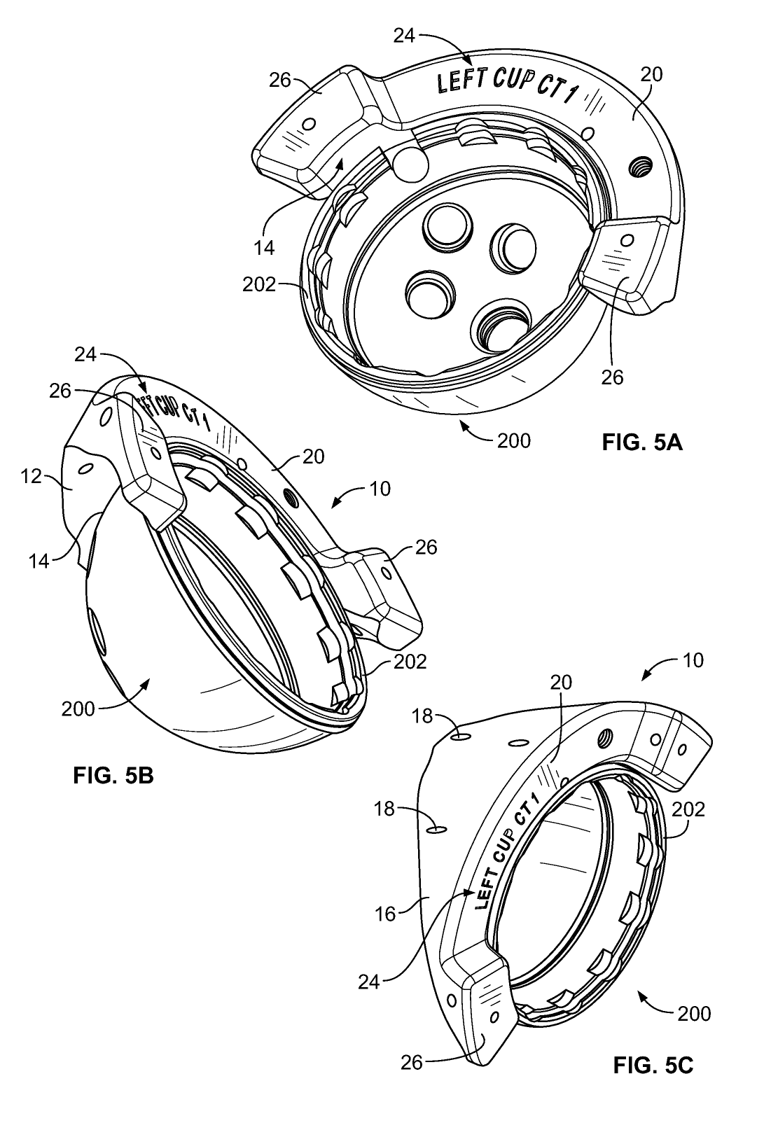

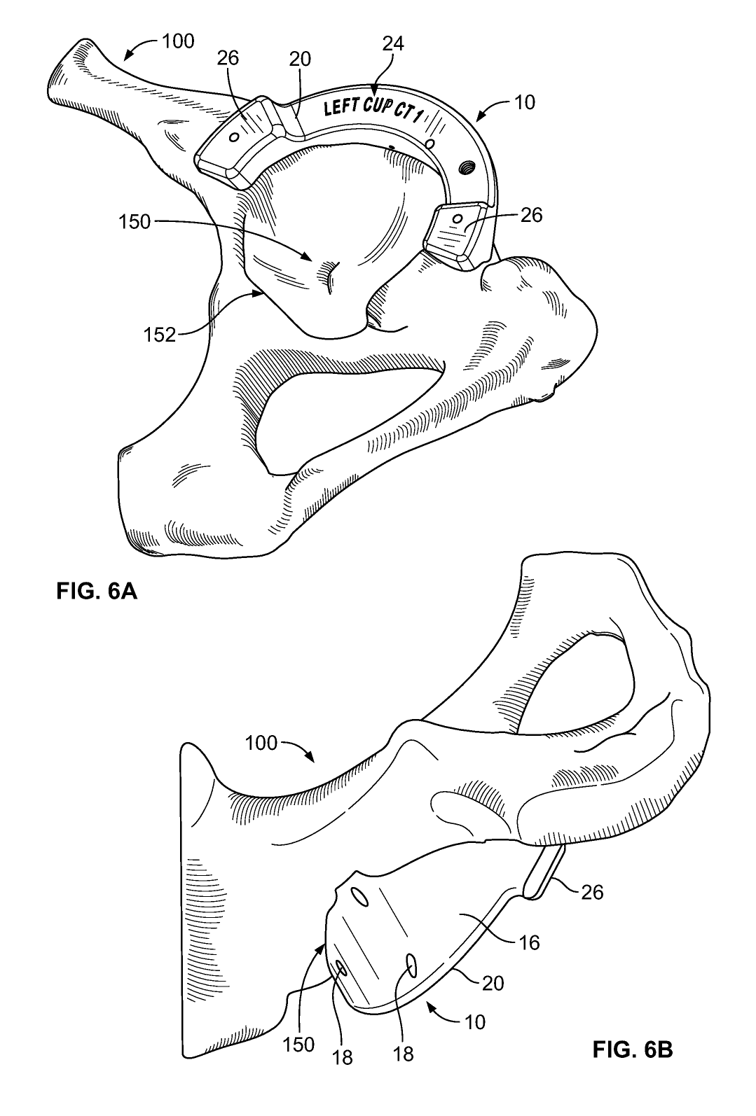

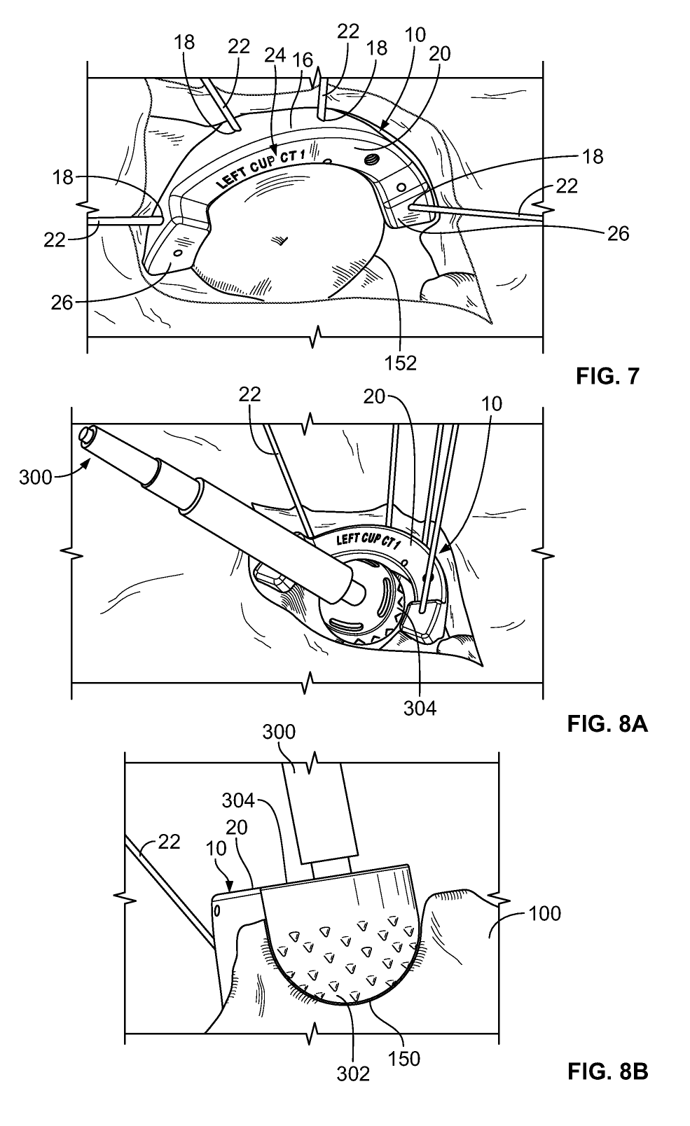

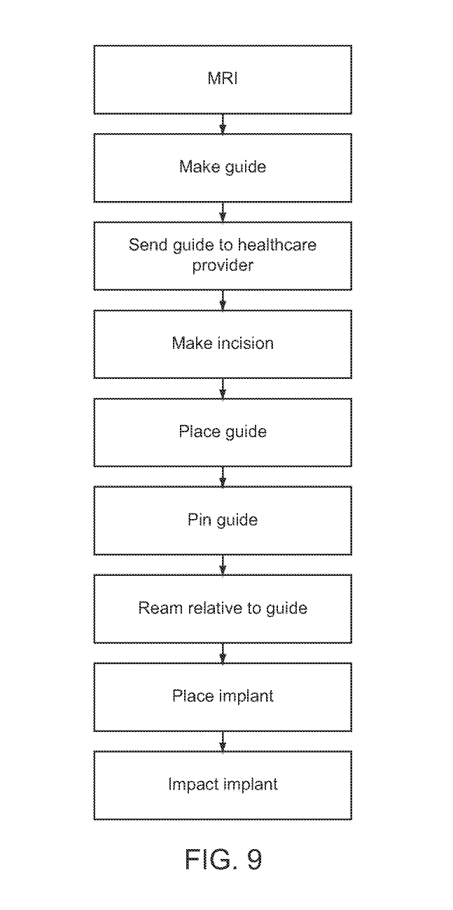

FIG. 1 shows an embodiment of a patient specific alignment guide 10 positioned on a pelvis 100, which includes acetabulum 150. FIGS. 2A-2D illustrate an embodiment of guide 10. FIG. 3 illustrates a guide 10 with angle indicators 124. FIGS. 4A-4B illustrate a guide 10 with a plurality of raised surfaces 26a-c. FIGS. 5A-5C illustrate an embodiment of a patient specific alignment guide 10 with acetabular implant 200. FIGS. 6A-6B illustrate an embodiment of a patient specific alignment guide 10 positioned on pelvis 100 proximate acetabulum 150. FIG. 7 illustrates a patient specific alignment guide 10 positioned on a pelvis through an anterior approach incision. FIGS. 8A-8B illustrate a bone shaping device 300 used to perform surgery on a pelvis. FIG. 9 illustrates a flow diagram depicting a process for making and using the patient specific alignment guide to perform surgery.

As seen in FIG. 1, acetabulum 150 is recessed to accommodate the proximal femur in a ball and socket joint configuration. Although not shown, the acetabulum also includes a labrum and joint capsule. The labrum is a small band of soft tissue surrounding the rim 152 of acetabulum 150 that acts like a bumper to prevent direct contact between the acetabular rim 152 and the neck of the proximal femur. The joint is a tunnel or envelope of soft tissues extending from acetabular rim 152 to the proximal femur. The joint capsule can visually or physically obstruct acetabular rim 152 during hip or other surgeries. The embodiment of guide 10 shown in FIG. 1 has a minimal footprint to conserve the labrum, capsule, and other soft tissue or cartilage while still effectively guiding the bone shaping device or acetabular implant 200.

FIGS. 2A-2D depict a surgical guide, adapted to the anatomy of a patient and configured to align a bone shaping device or acetabular implant in a predetermined orientation. Guide 10 includes an inner surface 12, a receiving portion or surface 14, a reference surface 20, and an outer surface 16. Guide 10 can be made of any appropriate polymer, plastic, medical grade nylon, or any metal to be sufficiently rigid to guide the bone shaping device or acetabular implant into the acetabulum. Inner surface 12, receiving portion 14, outer surface 16, and reference surface 20 of guide 10 can be made from the same or different materials.

Guide 10 has an inner surface 12, which is configured to abut at least a portion of the patient's unique hip anatomy in a single spatial position and orientation. The inner surface 12 has a contour that conforms to at least a portion of a specific patient's anatomy. This portion of the guide is generally a negative or complement of the topography of portions of the pelvic bone, cartilage, or other anatomy of a specific patient in which the guide fits.

In some embodiments, inner surface 12 is a patient-matched surfaces formed using topography data obtained from the specific patient by imaging devices. Computer aided design (CAD) and manufacturing techniques may also be used. For example, the topography of the patient anatomy, such as rim 152 of acetabulum 150, may be determined from X-ray, computerized tomography (CT) scans, magnetic resonance (MRI) scans, high definition ultrasound imaging, or other sources of information about the patient's bone and anatomical topography. In some embodiments, different MRI protocols may be executed on different patients. To minimize scan time, a fast spin echo imaging technique may be used for any protocol, essentially producing a proton density (PD) weighted image. Other imaging techniques or technologies may also be used to define and form the patient-matched surfaces. The patient topography data can be used to determine the shape of the inner surface 12. In practice, inner surface 12 conforms, or is complementary, to the patient's acetabular anatomy, such as rim 152.

Guide 10 includes a receiving portion or surface 14 that is configured to guide a bone shaping device (e.g., bone shaping device 300 depicted in FIGS. 8A-8B) or acetabular implant (e.g., implant 200 depicted in FIGS. 5A-5C) into acetabulum 150, as described herein. The receiving portion 14 may define an angle (e.g., abduction or anteversion angle) for directing an orthopedic device. For example, the receiving portion 14 may be angled to direct reaming and placement of an orthopedic implant at a target abduction (i.e., inclination) angle of 45° and a target anteversion angle of 20°. Other angles, as determined for a specific patient, may also be used. In certain embodiments, receiving portion 14 forms a smooth concave surface.

Guide 10 includes a reference surface 20 with an alignment site configured to serve as a visual and/or physical reference for guiding the bone shaping device 300 or acetabular implant 200. A plane defined by reference surface 20 may serve as an alignment site for the bone shaping device 300 or acetabular implant 200. In some embodiments, the plane defined by the position and/or orientation of reference surface 20 is substantially perpendicular to an axis defining a target abduction (inclination) and/or anteversion angles of a bone shaping device or acetabular implant. For example, the reference surface 20 may define a plane for the rim of an orthopedic bone shaping device or implant at a target abduction angle of 45° and a target anteversion angle of 20°. Other angles, as determined for a specific patient, may also be used. In some embodiments, the axis and/or plane is defined using the data relevant to the specific patient's pelvis.

The alignment site of reference surface 20 may also include indicia or indicators, such as instructive text, patient identifiers, positional information, angle markers, or depth markers. For example, the embodiment depicted in FIGS. 2A-2D includes indicia 24 which can be scored, marked, or otherwise formed on reference surface 20 of guide 10. In certain implementations, the indicia 24 may direct placement of the guide, for example to a specific surgical site or placement in a specific orientation. In FIGS. 2A-2D, for example, indicia 24 is located on reference surface 20 to indicate the proper location of guide 10. In some embodiments, the indicia 24 includes patient information or a unique patient identifier, such as a code, text, name, or location to help ensure the appropriate guide is used for a particular patient. Indicia 24 may placed on inner surface 12, outer surface 16, or receiving portion 14 of guide 10 to effectively guide the surgical process.

In some embodiments, the indicia 24 is used to help the surgeon reference varus, valgus, abduction, or anteversion angles and other navigational or directional planes or directions of interest. The surgical guide 10 illustrated in FIG. 3 has angle indicators 124, which may be used to reference varus or valgus angles. The angle indicators 124 may also be used as a reference for a desired reaming, impacting, or placement angle for an orthopedic device. For example, the angle indicators may define a target abduction angle of 45° and a target anteversion angle of 20°. As shown in FIG. 3, certain embodiments include a plurality of angle indicators 124. For example, the indicators 124 may provide a reference within a range of positions or angles. In certain embodiments the angle indictors are graduated, for example, at 1° increments.

In certain embodiments, the guide 10 has a raised or stepped surface 26 that is substantially parallel to reference surface 20. Raised surface 26 provides an additional visual guide and provides the surgeon more reference information. The difference in depth between raised surface 26 and reference surface 20 can indicate the desired reaming depth or implant 200 placement. For example, raised surface 26 may approximate the starting position of a device, such as bone shaping device 300, and reference surface 20 may approximate the target position, depth, or orientation of the device. Furthermore, raised surface 26 allows inner surface 12 of guide 10 to increase the area of contact with acetabulum 150 to further secure the guide 10 in place, help minimize contact, and protect bone or tissue in this region. As an example, after reaming, implant 200 is placed in the acetabulum 150, and the surgeon may use raised surface 26 to estimate the orientation of the implant 200 prior to impaction. The surgeon may also use reference surface 20 after impaction to estimate the resulting orientation of implant 200.

In certain embodiments, the reference surface 20 may have a plurality of steps or raised surfaces 26a-c as illustrated in FIG. 4 to aid in reaming the joint surface or placing the implant. The reference steps 26a-c may be used as depth indicators, where each step 26a-c corresponds to a predetermined depth. While FIG. 4 shows three steps 26a-c, any appropriate number of steps may be used. The steps, such as steps 26a-c, may also be used to guide different procedures of the surgery. For example, a first step, one of 26a-c, may correspond to the predetermined reamer orientation and a second step, one of 26a-c, may correspond to the predetermined implant orientation. Although FIGS. 4A and 4B show reference steps 26a-c and reference surface 20 in a substantially parallel configuration, in certain embodiments, the orientation angle of each step is unique and independent from the other steps in order to guide the orthopedic devices during different procedures (reaming, impacting, implant placement) of the orthopedic surgery.

FIGS. 5A-5C illustrate an embodiment of a patient specific alignment guide 10 with acetabular implant 200. The implant 200 can be guided along receiving portion 14 such that the rim 202 of orthopedic implant 200 is positioned and oriented at a desired position or orientation relative to reference surface 20 of alignment guide 10. For example, the desired position and/or orientation of the rim 202 or other reference surface of the acetabular implant 200 can be substantially co-planar or substantially parallel to a plane defined by reference surface 20. The plane defined by reference surface 20 can also relate to a desired insertion depth of acetabular implant 200. As will be described herein, receiving portion 14 and reference surface 20 may also be used to guide an orthopedic bone shaping device, such as device 300, for processes including, but not limited to, reaming and impacting.

In certain implementations, the rim 202 of the acetabular implant 200 or indicia on a bone shaping device can be placed parallel to reference surface 20. The angle of the rim 202 of acetabular implant 200 with respect to the flat reference surface 20 indicates the angle of implant 200. The physician can choose to align the plane of the rim 202 of implant 200 with the reference surface 20 or can angle the implant 200 at any desired angle with respect to reference surface 20. Reference surface 20 provides a visual indication of such angle.

FIGS. 6A-6B illustrate an embodiment of a patient specific alignment guide 10 positioned on pelvis 100 proximate acetabulum 150. The patient topography data can be used to determine the shape of the inner surface 12. In practice, inner surface 12 conforms, or is complementary, to the patient's acetabular anatomy, such as rim 152. Because inner surface 12 is configured, in some embodiments, to fit the patient's acetabulum 150, other cartilage, and soft tissue, the guide 10 is confined to one location and orientation within the patient's acetabulum. After insertion into the acetabulum, the guide 10 can be adjusted until the anatomy contacting portions of inner surface 12 align with the patient's acetabular anatomy, indicating, visually and/or tactually, that the guide 10 is in the proper position and orientation. After guide 10 is placed at the patient's acetabulum 150 by matching surface 12 to the anatomy, guide 10 may be further secured to the patient's acetabulum 150 using openings 18 and reference pins 22. The outer surface 16 of guide 10 preferably includes a plurality of openings 18. As depicted in FIG. 7, each opening 18 is configured to receive a fastener such as reference pin 22 or other securing device. Pins 22 may be used to removably fix or fasten the guide to the pelvic bone and/or cartilage. In certain embodiments, the openings can be on other surfaces. For example, reference surface 20 can also include openings 18, which can receive reference pin 22 or some other fastening device. In some embodiments, these structures are optional and do not necessarily have to be incorporated into guide 10.

Openings 18 may be angled relative to the bone. The axes of openings 18 may align with the intended incision allowing openings 18 to be visible or otherwise accessible when guide 10 is in place at the acetabulum 150. Because openings 18 are accessible, placing reference pins 22 through openings 18 may be easier in some instances than placing other fasteners such as screws, clamps, and conventional pins (not patient-matched). Reference pins 22 can be used to further fix or fasten the guide to the patient's hip. Further, openings 18 are configured to ensure that pins 22 are not obstructing bone shaping device 300 or acetabular implant 200 during insertion. The configuration of openings 18 also can orient reference pins 22 to help visually guide bone shaping device 300 or acetabular implant 200 into the pelvic area. Reference pins 22 can be used to establish a visual path for bone shaping device 300 or acetabular implant 200. It is understood that other embodiments can include different openings 18 and pins 22 depending upon surgical approach.

The position and/or orientation of the openings 18 and corresponding reference pins 22 provide additional functions, such as serving as retractors to maintain the incision site open. In conventional procedures, skin retractors are used to hold skin flaps around the surgical site. These conventional retractors are commonly scissor-like clamps with several limitations. For example, they are typically large and may fall off if bumped or otherwise need adjusted during the surgery. When acting as skin retractors, reference pins 22 are sufficiently long to extend beyond the patient's body when guide 10 is inserted so that pins 22 retain the skin in a retracted position to maintain an open incision site. The pins 22 can open the entire surgical site to provide an improved line-of-vision for the surgeon throughout the procedure. The placement and orientation of the openings 18 and pins 22 can be patient-matched, and can be placed according to the surgeon's preferences. The pins 22 can have a sharpened tip to facilitate insertion into the acetabulum 150. The pins 22 can be formed of metal or plastic, or any material sufficiently rigid to secure the guide into the acetabulum 150.

The location, orientation, and angle of bone shaping device 300 and the acetabular implant 200 are often critical to the success of a hip surgery. In certain embodiments the orthopedic device, such as bone shaping device 300 or acetabular implant 200, has a predetermined target orientation relative to the patient's anatomy. FIGS. 8A-8B illustrate a bone shaping device 300 used to perform surgery on a pelvis. Guide 10 is adapted to the anatomy of a patient and configured to align a bone shaping device or acetabular implant in a predetermined orientation for improved clinical outcomes. Once guide 10 is fixed or fastened to acetabulum 150, receiving portion 14 can guide a bone shaping device or acetabular implant by positioning and orienting the device or implant properly with respect to the acetabulum, and providing a visual and physical reference to the depth and angle of the guide or device, for example, by referencing reference surface 20, indicia 24, angle indicators 124, surface 26, or steps 26a-c.

Receiving portion 14 can physically constrain the device or implant and can guide the device or implant visually while not physically interfering with the device or implant. As a physical restraint, receiving portion 14 can limit the movement and depth of bone shaping device 300 as it is inserted into acetabulum 150 for reaming. Receiving portion 14 can physically limit the depth of cut by physically restraining contact between bone shaping device 300 and acetabulum 150. The bone shaping device 300 can contact the surface of receiving portion 14 to prevent a deeper cut. Further, receiving portion 14 can also limit side-to-side movement of bone shaping device 300 thus protecting the acetabular rim 152 from unintentional reaming. In this way, receiving portion 14 may also guide the bone shaping device 300 or implant 200 in a predetermined angle. In preferred embodiments, receiving portion 14 only contacts a portion of the bone shaping device 300 to allow freedom to ream as the surgeon determines appropriate. The guide does not limit bone shaping device 300 such that the surgeon cannot make calculated decisions or adjustments during the procedure regarding the depth and location of reaming. Receiving portion 14 forms a smooth concave surface.

Visually, receiving portion 14 can be used as a target when inserting bone shaping device 300 or acetabular implant 200 into the patient. Receiving portion 14 can be visible to guide the location of the device or implant. Further, during insertion, the physician can feel the receiving portion to determine the location of the acetabulum. Receiving portion 14 can have a smooth surface, so that the device or implant can slide into the acetabulum to any desired depth.

Guide 10 also includes a reference surface 20 with an alignment site configured to serve as a visual and/or physical reference for guiding the bone shaping device 300 or acetabular implant 200. Reference surface 20 of guide 10 provides additional reference information for placing a bone shaping device or implant into the acetabulum. Reference surface 20 visually assists navigation of the bone shaping device 300 or acetabular implant 200 into the acetabulum 150. Reference surface 20 can have a flat surface on guide 10 that is perpendicular to the desired direction and trajectory of bone shaping device 300 or acetabular implant 200. Thus, as bone shaping device 300 or acetabular implant 200 is inserted into the acetabulum, the reference surface 20 can assist in guiding the device 300 or implant 200 to an appropriate location and angle of insertion. The physician can look at the reference surface and determine an angle to ream the acetabulum or an angle to insert the implant. For example, the reference surface may have angle indicators, such as indicators 124, to guide alignment. After insertion into the acetabulum, the guide 10 can be adjusted until the anatomy contacting portions of inner surface 12 align with the patient's acetabular anatomy, indicating, visually and/or tactually, that the guide 10 is in the proper position and orientation.

The bone shaping device 300 or acetabular implant 200 can be guided such that a rim or other reference surface on the bone shaping device 300 or acetabular implant 200 is positioned and oriented to reflect a desired position or orientation relative to reference surface 20 of alignment guide 10. A plane defined by reference surface 20 may serve as an alignment site for the bone shaping device 300 or acetabular implant 200. For example, the desired position and/or orientation of the rim or other reference surface on bone shaping device 300 or acetabular implant 200 can be substantially co-planar or substantially parallel to a plane defined by reference surface 20. The plane defined by reference surface 20 can also relate to a desired reaming or insertion depth of bone shaping device 300 or acetabular implant 200. In some embodiments, the plane defined by the position and/or orientation of reference surface 20 is substantially perpendicular to an axis defining desired inclination and/or anteversion angles of bone shaping device 300 or acetabular implant 200. For example, the reference surface 20 may define a plane for the rim of an orthopedic bone shaping device or implant at a target abduction angle of 45° and a target anteversion angle of 20°. Other angles, as determined for a specific patient, may also be used. In some embodiments, the axis and/or plane is defined using the data relevant to the specific patient's pelvis.

With respect to depth of reaming and depth of the implant into the acetabulum, reference surface 20 provides an established level that can be pre-determined based on the patient's anatomy. Throughout the bone shaping process, the surgeon may tactually feel the depth of the reaming device 300 and compare that to the reference surface 20. The cutting portion 302 of the reamer contacts and shapes the acetabulum 150. The surgeon makes tactile contact with the reference surface 20 and the rim 304 to determine the depth of the reaming. For example, the surgeon may place one finger on the reference surface 20 and one finger on the rim 304. The surgeon may also slide a finger across the reference surface 20 and rim 304 to determine the difference in position or depth. In some methods, the surgeon may stop reaming when a portion of the reamer feels as if it is flush with reference surface 20. The rim of implant 200 can be co-planar with the reference surface to indicate desired depth, or should the physician desire, the rim can fall below or remain above the reference surface as determined appropriate by the surgeon. Again, reference surface 20 provides a visual indicator of such depth. For example, the surgeon may need to remove bone spurs, sclerotic (hardened bone), or cysts. Throughout the reaming process, the surgeon may check to whether the reaming has removed this unhealthy bone. For example, bleeding from the bone can indicate healthy, vascularized bone that is able to receive and implant and grow to incorporate an implant, such as implant 200. The surgeon may check for healthy bone and adjust the reaming depth accordingly to be higher or lower than reference surface 20. In other embodiments, the surgeon may check the tightness or fit of the joint and adjust the reaming depth or orientation accordingly. The physician may also utilize other parts of guide 10, such as indicia 24, angle indicators 124, surface 26, or steps 26a-c as references to guide the bone shaping device 300 or implant 200 into position.

Controlling and adjusting the depth of the reaming in the revision acetabulum can be used to correct leg length discrepancies with the contralateral side. The reaming depth can be determined virtually based on matching the leg length of the contralateral side or a surgeon directed correction. In cases where large discrepancies exist, the surgeon may only want to correct a portion of it because the soft tissues cannot sufficiently stretch to the desired length. For example, in a 30 mm discrepancy, the surgeon may decide to only correct 15 to 20 mm. The surgeon may determine this adjustment and is it to appropriate define the features and surfaces of the guide. They surgeon also make adjustments during the surgery based on the progress of the procedure.

FIG. 9 illustrates a flow diagram depicting a process for making and using the patient specific alignment guide to perform surgery. The steps can include performing an MRI, making the patient matched guide based on data obtained from the MRI, sending the guide to a healthcare provider, having a surgeon make an incision on the patient, placing the guide in the acetabulum, pinning the guide, reaming relative to the guide, placing the implant relative to the guide, impacting the implant, and any combination or subcombination thereof.

Anatomic models of the acetabulum 150 and surrounding portions of the pelvic anatomy, including the labrum, joint capsule, and other soft tissue and cartilage may be extracted from MRI data or other images to identify appropriate anatomic reference landmarks. Full hip X-rays can be used to determine the mechanical axis alignment and/or total version. The guide 10 may then be designed and prepared through computer-aided design (CAD) modeling based on the anatomic model. For instance, in some embodiments, the anatomic model is used to define the structure of the guide 10 that conforms to the patient's anatomy to define the particular position and angle of the guide 10 relative to the patient's anatomy. The anatomic model, in conjunction with other data, may also be used to define the position, orientation and other aspects of the patient-matched inner surface 12 and the reference surface 20 on the guide, including alignment sites such as indicia 24, raised surface 26, steps 26a-c, and angle indicators 124. The anatomic model may also be used to determine appropriate placement and orientation of openings 18 for pins 22 to secure the guide 10 to the bone and, in certain embodiments, retract skin or other tissue during the procedure.

Other aspects of the guide may also be customized specifically for the patient. CAD and other automated or non-automated techniques, according to certain embodiments, can be configured to accommodate input or definition from the surgeon, if desired, on matters such as dimensions of various parts of guide 10, intended location for bone shaping device 300 or acetabular implant 200. The planned configuration may include angle, path, shape, dimensions, or other characteristics of the implants and devices. The planned configuration may also include the angle, path, shape, dimensions, or other characteristics of the resections to be performed or used in connection with guide 10. At least portions of the inner surface 12 of guide 10 may be made from any desirable material using, for example, laser techniques, such as the “EOSINT P” series machine or any other desired computer aided manufacturing system. EOSINT is a registered trademark of EOS GmbH Electro Optical Systems, Krailling, Germany. Because the inner surface 12 of guide 10 is based on the patient's data set, data which properly differentiates between bone and cartilage and soft tissue could be used to ensure the fit and functionality of guide 10.

In these and other embodiments, the anatomy-contacting portions of inner surface 12 and other portions of the guide can be customized using the data from patient imaging (e.g., MRI) that may include a compilation of 3D image data slices which are spaced by intervals of approximately 2-4 mm. Interpolation algorithms may be used to approximate anatomical geometries between the image data slices.

All embodiments of the guides shown in the figures may be used in any hip approach. The embodiment of guide 10 as shown in the figures is a guide that could be designed for a posterior-lateral surgical approach. However, it is understood that different embodiments of guide 10 may be used for different approaches, for example, the same or a different embodiment may be used in anterolateral, direct-lateral, posterolateral, direct-anterior, and any variations on these approaches. The profile of the guides is meant to minimize disturbance of soft tissue around acetabulum 150 and minimize disruptions to blood flow. While the embodiments have been disclosed individually, portions of each embodiment may be interchangeable and combined to form an alignment guide in an alternate embodiment.

As various modifications could be made in the constructions and methods herein described and illustrated without departing from the scope of the embodiments herein disclosed, it is intended that all matter contained in the foregoing description or shown in the accompanying drawings shall be interpreted as illustrative rather than limiting. In view of the foregoing, it will be seen that the several advantages of the various embodiments are achieved and attained. Thus, the breadth and scope of these embodiments should not be limited by any of the above-described exemplary embodiments, but should be defined in accordance with the claims appended hereto and their equivalents.

The foregoing is merely illustrative of the principles of the disclosure, and the systems, devices, and methods can be practiced by other than the described embodiments, which are presented for purposes of illustration and not of limitation. It is to be understood that the systems, devices, and methods disclosed herein, while shown for use in acetabulum or hip systems, may be applied to systems, devices, and methods to be used in other surgical procedures including, but not limited to, knee procedures, spine arthroplasty, cranio-maxillofacial surgical procedures, shoulder arthroplasty, as well as foot, ankle, hand, and extremities procedures.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

1. An orthopedic surgical guide, comprising:
a first surface structured to fit in a predetermined configuration relative to a portion of a patient's anatomy based on topography data of the patient's anatomy;
a receiving surface structured to constrain a portion of an orthopedic device;
a reference surface having an alignment site that directs placement of the orthopedic device in a predetermined orientation relative to the patient's anatomy when the first surface is in the predetermined configuration.

2. The surgical guide of claim 1, wherein the first surface has a contour that conforms to the portion of a patient's anatomy.

3. The surgical guide of claim 1, wherein the topography data is obtained from an imaging device.

4. The surgical guide of claim 1, wherein the alignment site is positioned based on the topography data, and wherein placement of the alignment site in a predetermined position aligns the guide in the predetermined configuration.

5. The surgical guide of claim 1, wherein the alignment site includes an indicator that is scored, marked, or integrally formed with respect to the reference surface.

6. The surgical guide of claim 5, wherein the indicator identifies a predetermined anatomical location for placement of the surgical guide.

7. The surgical guide of claim 5, wherein the indicator includes a unique patient identifier.

8. The surgical guide of claim 1, wherein the predetermined configuration corresponds to a predetermined depth of the orthopedic device relative to the patient's anatomy.

9. The surgical guide of claim 8, wherein the reference surface lies in a plane parallel to a surface of the orthopedic device when the orthopedic device is placed in the predetermined orientation.

10. The surgical guide of claim 8, wherein the reference surface comprises a plurality of steps, each step corresponding to a different predetermined depth of the orthopedic device.

11. The surgical guide of claim 10, wherein a first step corresponds to a predetermined depth for positioning a reamer and a second step corresponds to a predetermined depth for positioning an implant.

12. The surgical guide of claim 1, wherein the predetermined configuration comprises a predetermined angle of the orthopedic device relative to the patient's anatomy.

13. The surgical guide of claim 12, wherein the predetermined angle is one of a varus or valgus angle.

14. The surgical guide of claim 12, wherein the alignment sites corresponds to the predetermined angle.

15. The surgical guide of claim 12, wherein the receiving surface corresponds to the predetermined angle.

16. The surgical guide of claim 1, wherein the receiving surface is concave.

17. The surgical guide of claim 1, further comprising an opening structured to receive a fastener.

18. The surgical guide of claim 17, wherein the location of the opening is based on the topography data.

19. The surgical guide of claim 1, wherein the surgical device is one of a reamer, an impactor, and an acetabular cup.

20. A method for making a surgical guide, comprising:
obtaining data indicative of a patient's anatomy;
creating a model of the patient's anatomy from the data;
determining a suitable position for an orthopedic device relative to the model;
forming a first surface structured fit in a predetermined configuration relative to the patient's anatomy based on the model;
forming a receiving surface structured to contact a portion of an orthopedic device;
forming a reference surface with an alignment site that directs placement of the orthopedic device in a predetermined orientation relative to the patient's anatomy.

21. The method of claim 20, further comprising forming at least one opening based on the model, wherein the opening is structured to receive a fastener.

22. The method of claim 20, further comprising forming a contour on the first surface, wherein the contour is complementary to a portion of the patient's anatomy.

23. A method of placing an orthopedic device, comprising
providing a surgical guide having a first surface, receiving surface, and reference surface with an alignment site that directs placement of the orthopedic device in a predetermined orientation relative to the patient's anatomy, wherein the first surface is structured to fit in a predetermined configuration relative to a portion of a patient's anatomy based on topography data of the patient's anatomy;
placing the first surface on the patient anatomy in the predetermined configuration;
mating the orthopedic device with the receiving surface; and
aligning a first portion of the orthopedic device with the reference surface.

24. The method of claim 23, wherein the surgical guide has an opening, and further comprising inserting a pin into the opening to secure the guide to bone or tissue.

25. The method of claim 24, further comprising retaining skin tissue in a retracted position with the pin.

26. The method of claim 23, further comprising verifying the depth of the orthopedic device by comparing the position of the first portion of the device to the position of the alignment site of the guide.

27. The method of claim 26, further comprising aligning the first portion of the orthopedic device with the alignment site of the guide.

28. The method claim 26, wherein verifying the depth of the orthopedic device further comprises making tactile contact to the first surface of the device and making tactile contact to the reference surface of the guide.
